(19) United States
(12) Patent Application Publication
(10) Pub. No.: US 2011/0125108 A1
(43) Pub. Date: May 26, 2011

(54) SURGICAL INSTRUMENT PREFERABLY WITH TEMPERATURE CONTROL

(75) Inventors: Jacques Deviere, Genappe (BE); Alain Delchambre, Bruxelles (BE); Nicolas Cauche, Bruxelles (BE); Sonia Dugardeyn, Braine le Comte (BE); Philippe Dubois, Braives (BE); Jean-Marie Raquez, Mons (BE)

(73) Assignees: Universite Libre de Bruxelles, Bruxelles (BE); Universite de Mons, Mons (BE)

(21) Appl. No.: 12/992,248

(22) PCT Filed: May 18, 2009
(86) PCT No.: PCT/EP2009/056030

§ 371 Date: Jan. 10, 2011

(30) Foreign Application Priority Data
May 16, 2008 (EP) 08156402.3
May 16, 2008 (EP) 08156403.1
Nov. 19, 2008 (EP) 08169414.3

(51) Int. Cl. A61M 5/32

(52) U.S. Cl. 604/272

(57) ABSTRACT

A surgical needle has shape memory. An endoscope tool controls the temperature of a foreign body or surgical instrument such as an endoscopic needle.

### FIELD OF APPLICATION

The present invention concerns the field of surgical instruments.

In particular, according to a first aspect, the present invention concerns a surgical needle, and in particular an endoscopic needle whereof the access to the site to be treated is by an endoscopy route.

The present invention also concerns the device making it possible to use said surgical needle.

Secondarily, the present invention concerns, according to a second aspect, the temperature control of a surgical instrument and in particular a therapeutic instrument, a diagnostic instrument or an implant.

Preferably, the temperature control of the instrument or implant is done, in situ, inside the human body or the animal's body.

Advantageously, the access of the therapeutic instrument, diagnostic instrument, or implant which constitutes a foreign body at the site to be treated is done by an endoscopy route.

Lastly, the present invention concerns a therapeutic treatment making it possible to use such surgical needles that may be associated with a temperature control tool.

### TECHNICAL PROBLEM

During a surgical operation, when it is necessary to perform a suture of a mucosa such as a suture through a wall like a purse-string [en bourse] suture, a continuous suture, a plication, or an anastomosis, that is located inside the human body, the surgeon uses a rigid and sharp curved needle. The curved shape of this needle allows him to pass the needle, through a rotational movement, to the other side of the wall and bring it back without having to fold [said] this wall. The suture can only be done on the condition that the curved needle can be brought onto the site to be treated and can be manipulated with the different degrees of freedom necessary to perform the suture.

In certain [flexible] supple endoscopy interventions such as, for example, treating a significant gastric hemorrhage requiring a suture within the gastric cavity, these two conditions are not met. The gastroenterologist cannot currently bring a curved needle towards the gastric cavity because the natural access routes to the gastric cavity are too narrow or too sinuous. Because of this, when a suture must be done within the gastric cavity, the patient must undergo a heavy surgical intervention that must allow all of the tissues to be opened to reach the organ in question, which considerably increases the traumatism, and especially for obese patients or patients with third degree burns for whom cicatrization of the skin is delicate. This constitutes one of the major limitations of the [flexible] supple therapeutic endoscopy done using natural routes.

Several solutions to perform sutures have been proposed and can be classified as follows.

A first category comprises sutures that are done by making a fold—for example by sussion—and passing a straight needle into the fold. For example, these interventions are used to form plications situated at precise locations of the gastric cavity or the low esophagus.

Specifically, to treat gastro-esophageal reflux, several devices allow to perform a plication of the cardia (NDO®, Plicator®; Bard Therapeutic®, Endocinch®) or to place a sleeve [un manchon] around the cardia. These techniques use a sophisticated material, either attached to the extremity [au bout de] of the endoscope, or consisting of a complex instrument in which an endoscope is introduced. The aim of these devices is to offset [pallier] the fact that the endoscopist is a surgeon with only one hand who cannot pull on the tissue to make a suture. These devices aiming to plicate the cardia make it possible to grasp the entire gastric wall and suture it to another part of said wall.

Another category is related to the creation of anastomoses (gastroenterostomy) between the stomach and the small [grêle] intestine, where a suture of both walls (seroserous) is necessary. Certain techniques are currently being developed (T Tags, T bars), but only allow periodic [ponctuelles] sutures and cause [induisent] a risk a puncturing [ponctionner] adjacent organs when straight and rigid needles are used. Performing a continuous [to ensurjet] suture is not possible in that case.

Several documents of the prior art describe devices allowing to bring a curved needle inside a patient's body. In particular, documents WO-9508296 and EP-0529675 describe a needle that can make a transition between two stable forms at two different temperature ranges.

In document WO-9508296, the needle is made in a shape memory alloy [alliage]. The shape of the endoscopic needle is modified by the surrounding temperature such that at ambient temperature (defined in this document as being between 0° C. and 24° C.), the needle is straight and at a temperature between 25° C. and 40° C., the needle becomes curved.

In document EP-0529675, the needle is made of a shape memory alloy [alliage]. The shape of the endoscopic needle is modified by outside heat sources such as “illumination light,” “laser,” or “cautery” such that the needle is straight at a temperature below 25° C. and the needle is curved at a temperature above 35° C.

The needles described in the two documents cited above are not as rigid as the curved needles typically [habituellement] used by surgeons. In fact, in the examples cited in these two documents, the needles in their curved shape are removed from the patient's body by applying a mechanical stress [contrainte] via an intermediate tube.

For the needles described in the two aforementioned documents to change shape, they must be exposed to a heat source over a large portion of their outer surfaces. These needles can only be used in their curved form in the body. This limits the therapeutic possibilities in relation to a needle that could be used both in its curved form and in its rectilinear form inside the human body.

The transition from a curved shape to a straight shape inside the human body would make it possible to increase suture possibilities while using a needle with a rigidity equivalent to that offered for example by the needles of the “Ethilon® CPX 1(45 mm)” or “Ethilon® FS-3 (16 mm ⅜ c)” type provided by Ethicon® (Johnson&Johnson®).

To this extremity, it may be interesting to provide for the temperature control (cool or heat quickly) of a foreign body and in particular a surgical instrument such as a needle situated inside the human body and where of access is by endoscopy route.

This change in the temperature of the foreign body must be done while preserving the integrity of the tissues situated nearby and independently of the fact that the foreign body is in direct contact with the patient's body.

Among the different heating devices, diathermanous needles are known for providing a ponctual heat source to a tissue. However, this principle cannot be applied to heat an instrument in the body while preserving the integrity of the tissue.

### MAIN CHARACTERIZING ELEMENTS OF THE INVENTION

A first object of the invention concerns a shape memory surgical needle comprising a needle body having two extremitiesextremities, distal and proximal, said body having at least two different stable shapes, or a first shape stable at a first temperature range and a second shape stable at a second temperature range, the first temperature range being strictly lower than the second temperature range; characterized in that the material making up the body of the needle comprises at least one shape memory insulating material and at least one heat and/or electrical conductor material; the entire body of the needle being capable of being heated in situ by [en] applying for example a heat and/or current supply. The temperature variation caused by the heat and/or current supply in the needle allows the transition from the first stable shape to the second stable shape and this inside the body.

Insulating material refers to a polymer matrix having insulating properties.

Preferably, this polymer matrix assumes the form of a copolymer multiblocks that comprises at least two types of blocks.

Preferably, a first type of blocks has a low softening temperature and a second type of block has a high softening temperature.

Preferably, the softening temperature of the first type of blocks is strictly lower than the softening temperature of the second type of blocks.

Block here refers to a uniform sequence of monomers, forming a homopolymer or a uniform statistical copolymer.

Softening temperature refers either to a vitreous transition temperature, or a melting temperature.

Preferably, the softening temperatures correspond to melting temperatures.

Preferably, each of the types of blocks has at least two terminal alcohol functions.

Preferably, the multiblocks are obtained as a reaction product of several blocks each comprising at least two terminal alcohol functions with diisocyanates.

More particularly, an insulating-type polymer results from the reaction of a first polymer (block) and a second polymer (block) with a diisocyanate in which both the first and the second polymers comprise terminal alcohol functions.

Preferably, the first polymer has a softening temperature strictly lower than that of the second or secondary polymer.

Preferably, the softening temperature of the first polymer is between 38° C. and 60° C.

Preferably, the softening temperature of the second polymer is at least 10° C. higher than that of the first polymer.

Preferably, the softening temperature of the second polymer is at least 20° C. higher than the softening temperature of the first polymer.

Preferably, the softening temperature of the first polymer is the transition temperature between two stable shapes of the needle (switching temperature).

Preferably, the first polymer and the second polymer form separate phases, preferably co-continuous.

Preferably, the polymers are selected among biocompatible polymers.

Preferably, these polymers are biocompatible according to standard ISO 10993-1:1997.

Preferably, the two polymers are selected from the group consisting of aliphatic polyesters, aliphatic polycarbonates, aliphatic polyester-alt-ethers, poly(ethylene glycol) and all combinations thereof.

Preferably, the two polymers are chosen from the group consisting of polycaprolactone, poly(para-dioxanone), poly(ethylene glycol), statistical copolymer of polycaprolactone-polylactide, statistical copolymer poly(para-dioxanone-caprolactone), statistical copolymer of polycaprolactone-polyglycolide, statistical copolymer of polycaprolactone-polylactide-polyglycolide, polylactide, statistical copolymer of polycaprolactone-poly(.beta.-hydroxybutyric acid), poly(.beta.-hydroxybutyric acid), and combinations thereof.

Preferably, the diisocyanate is selected from the group consisting of 4,4′-diphenyl methylene diisocyanate, toluene-2,4-diisocyanate, toluene-2,6-diisocyanate, hexamethylene-1,6-diisocyanate, isophorone diisocyanate, hydrogenated 4,4′-diphenylmethane diisocyanate and mixtures thereof.

Preferably, the multiblock polymer is made up of a statistical or regular alternation of polycaprolactone-type blocks, having a melting temperature of about 40 to 45° C., and blocks of a statistical copolymer of caprolactone and polyester-alt-ether such as poly(-para-dioxanone) having a melting temperature between about 60 and 70° C.

The multiblock polymer can have different architectures going from linear to star structures.

Preferably, the thermic and/or electrical conductor material can be an electrical connection.

Preferably, the thermic and/or electrical conductor material is a thermic and/or electrical conductor wire.

Preferably, the thermic and/or electrical conductor material assumes the form of a thermic and/or electrical conductive charge submerged within the polymer matrix, said charge being present at a concentration above its percolation threshold.

Percolation threshold refers to the concentration from which the charge forms a continuous conducting network.

Preferably, the thermic and/or electrical conductor charge assumes the form of powder, fiber or sheets with dimensions preferably between about 10 μm and 5 nm.

Preferably, the conductor charge will be selected from the group consisting of carbon blacks, carbon fibers, carbon nanofibers, carbon nanotubes, grapheme lamellas (exfoliated graphite), or a mixture thereof.

Insulating refers to a resistivity greater than 10⁸ Ohm·m while conductor refers to a resistivity less than 10³ Ohm·m.

Stable shape, refers to a geometric shape that remains essentially unchanged for temperatures belonging to a temperature range.

Advantageously, this needle will not undergo shape or structure modification if light mechanical forces are applied to it in the order of 10 N that correspond to a human manipulation.

Preferably, this needle will not undergo shape or structure modification if light mechanical forces are applied to it in the order of 5 N that correspond to a human manipulation.

Preferably, the first stable shape is rectilinear.

Preferably, the second stable shape is curved.

Preferably, the first stable shape is a rectilinear shape and the second stable shape is a curved shape of the needle.

Preferably, the entire body of the needle is suitable for being cooled by applying a cold source to it.

Preferably, the temperature variation caused by the cold source in the needle allows the transition from the second stable shape at a second temperature range to a third stable shape at a third temperature range.

Advantageously, this third stable shape corresponds to the first stable shape.

Alternatively, the temperature of the needle is brought to a temperature above the softening temperature (high) of the secondary or second polymer and belonging to a fourth temperature range, before cooling it to the third temperature range.

Preferably, a mechanical stress is exerted on the needle when it has reached the fourth temperature range.

Alternatively, the temperature variation caused by the cold source in the needle allows the transition from the second temperature range to the third temperature range while preserving the second stable shape and increasing the mechanical properties of the needle.

Preferably, said thermic and/or current supply is in direct contact with one of the extremities, preferably the proximal extremity of the needle.

Preferably, said cold source is in direct contact with one of the extremities, preferably the proximal extremity of the needle.

Preferably, at least one of the extremities, and preferably the proximal extremity of said needle, is made of a thermic and/or electrical conductor material that can be different from or identical to the thermic and/or electrical conductor material making up the body of the needle.

Said proximal extremity is preferably in direct contact with the conductor material contained in the rest of the needle.

Preferably, the shape of the needle in the first temperature range is rectilinear.

Preferably, the shape of the needle in the second temperature range is curved.

Preferably, the shape of the needle in the third temperature range is rectilinear.

Advantageously, the transition between the first and second stable shapes is done at a first switching temperature Ts₁.

Advantageously, the transition between the second and third stable shapes is done at a second switching temperature Ts₂.

Particularly advantageously, the first switching temperature corresponds to the second switching temperature.

Advantageously, the first temperature range is strictly lower than the first switching temperature.

Advantageously, the third temperature range is strictly lower than the second switching temperature.

Advantageously, the second temperature range is strictly higher than the first and the second switching temperatures.

Advantageously, the transition between said first and second shapes occurs at a switching temperature Ts₁ between a minimum temperature corresponding to a temperature just above body temperature, that is to say about 38° C., and a maximum temperature of about 60° C., preferably at a switching temperature Ts₁ between 40° C. (maximum temperature of the human body) and 45° C.

Advantageously, the transition between the second and third embodiments occurs at a switching temperature Ts₂ between a minimum temperature just above body temperature about 38° C. and a temperature of about 60° C. and preferably between 40° C. and 45° C.

Preferably, below its switching temperature(s), the needle is rectilinear.

Preferably, above its switching temperature(s), the needle is curved.

Preferably, when the temperature is strictly below a temperature just above body temperature (38° C.), the needle is in its rectilinear shape.

Preferably, when the temperature is strictly above 45° C. the needle is in its curved shape.

Preferably, the distal extremity of the needle is sharp.

Preferably, the distal extremity of the needle has a metallic or ceramic insert making it possible to ensure the integrity of its sharpness.

Preferably, the proximal extremity of the needle receives a surgical wire.

A second aspect of the present invention concerns an endoscopic tool intended to control the temperature of a foreign body or surgical instrument such as an endoscopic needle and in particular the needle described above. This tool is made up of at least three parts: a distal extremity—extremity penetrating in the human or animal body—, the—flexible or rigid—body of the tool and a proximal extremity—extremity staying outside the human or animal body.

The tool is designed so that the distal extremity of the tool comes into contact with the foreign body.

The distal extremity of the tool is capable of fastening to the foreign body, such as a needle, to be heated (or to be cooled).

Preferably, the distal extremity has at least one conductor part—electrical and thermic—that can be heated and cooled in a controlled manner.

Advantageously, this conductor part is part of the contact interface with the foreign body.

Advantageously, this conductor part is in contact with the foreign body.

The shape of this conductor part is preferably adapted to optimize the contact surface with the foreign body so as to optimize the thermic transfer by conduction.

Preferably, the conductor part(s) are connected to two conductor connections insulated from the rest of the tool and its environment.

Preferably, these two connections assume the shape of wires making it possible to form a closed current loop through the conductor part(s) with or without the foreign body.

Preferably, the current passing through this or these conductor part(s) also passes in the foreign body and heats it by Joule effect. The conductor part of the tool, according to the second aspect of the invention, is then heated by conduction.

Alternatively, the current passing in the conductor part(s) heats this (these) conductor part(s) by Joule effect and the foreign body is then heated by conduction.

Alternatively, the two aforementioned manners are combined.

The conductor part(s) can be cooled by spraying them or by putting them in contact with a refrigerated fluid preferably coming preferably from an inlet tube whereof the distal extremity preferably coincides with the distal extremity of the tool according to the second aspect of the invention.

Preferably, the refrigerated fluid is sprayed or is in contact with the foreign body and the foreign body is then cooled by convection. The conductor part of the tool according to the second aspect of the present invention is then cooled by conduction.

Alternatively, the refrigerated fluid is sprayed or in contact with the conductor part(s) that are then cooled by convection and that then cools the foreign body by conduction.

Alternatively, the two aforementioned manners are combined.

Preferably, the conductor part(s) are, with the exception of the two connections, thermally and electrically insulated from the rest of the tool according to the second aspect of the invention.

Preferably, at least one temperature sensor is present on one of the conductor parts of the tool according to the second aspect of the invention. This sensor makes it possible to measure the temperature of the foreign body and is intended to allow temperature control.

Preferably, this temperature sensor is a thermocouple.

The distal extremity of the tool can be a clip.

Preferably, the distal extremity of the tool comprises a clip.

The distal extremity of the tool can be a clip with at least two interior jaws.

Preferably, the distal extremity of the tool comprises a clip with at least two interior jaws.

The distal extremity of the tool can be a clip with at least two exterior jaws.

Preferably, the distal extremity of the tool comprises a clip with at least two exterior jaws.

The distal extremity of the tool can coincide with the extremity of a catheter.

The distal extremity of the tool can be an inflatable balloon.

Preferably, the distal extremity of the tool comprises an inflatable balloon.

The distal extremity of the tool can be a vacuum.

Preferably, the distal extremity of the tool comprises a vacuum.

The distal extremity of the tool that serves for temperature control can correspond to the distal extremity of most of the traditional instruments used in endoscopy.

Preferably, the body of the tool according to the second aspect of the invention is designed to make it possible to place the distal extremity of said tool at the site to be treated. The body of the tool is designed in relation with the functionalities of the distal part of said tool.

For example, in the case where the distal extremity is a clip with two jaws, the body of the tool comprises a means for transferring the force making it possible to open and close the clip.

This means for transferring the force between the distal extremity and the proximal extremity can, for example, in the case of an endoscopic clip, be a flexible metallic rod sliding in a flexible metallic sheath.

Preferably, the body of the tool comprises at least one transport means (tube(s) and/or electrical wires) for the energy necessary to cool and/or heat the conductor part(s).

Preferably, the energy transporting means is made up of an inlet duct [consistent] for a fluid (water or gas) making it possible to cool the foreign body and of two conductor connections making it possible to convey electrical current or thermic energy.

Preferably, the body of the tool comprises at least one means for transferring the temperature measurement.

This means for transferring the temperature measurement between the proximal extremity and the distal extremity can for example be two insulated conductor wires of a thermocouple.

Preferably, the proximal extremity of the tool is connected with the functionalities of the distal part of said tool.

Preferably, the proximal extremity of the tool comprises at least connectors necessary to connect the tool according to the second aspect of the invention to at least one energy source, in particular a first energy source (hot) and a second energy source (cold).

Preferably, the proximal extremity of the tool comprises at least one connector making it possible to connect the means for transferring the temperature measurement located in the body of the tool to a data acquisition system that is connected to a temperature controller.

Advantageously, the first energy source is a current supply.

Advantageously, the first energy source is a heat supply.

Advantageously, the first energy source is a current and heat supply.

Advantageously, the second energy source is a coolant fluid supply.

Advantageously, the second energy source is a cold water supply.

Preferably, one of the conductor connections previously defined makes it possible to provide the necessary energy to the foreign body to perform an electrocoagulation diathermy.

Preferably, a third conductor connection is provided to connect the conductor part of the tool according to the second aspect of the present invention so as to be able to provide the necessary energy to the foreign body to perform the electrocoagulation diathermy.

The present invention also concerns the use of the needle and/or of the tool for therapeutic or diagnostic interventions.

### BRIEF DESCRIPTION OF THE DRAWINGS

FIGS. 1a and 1b illustrate the three stable shapes of the needle—curved shape (stable shape 2 illustrated in FIG. 1a) and straight shape (stable shape 1 and 3 illustrated in FIG. 1b)—according to a first preferred embodiment of the first aspect of the present invention.

FIGS. 2a and 2b illustrate the three stable shapes of the needle—curved shape (stable shape 2 illustrated in FIG. 2a) and straight shape (stable shape 1 and 3 illustrated in FIG. 2b)—according to a second preferred embodiment of the first aspect of the present invention.

FIGS. 3a-c illustrate a needle according to different preferred embodiments of the first aspect of the present invention.

FIG. 4 illustrates a lateral view of the tool according to one preferred embodiment of the second aspect of the present invention, the tool comprising a clip with 2 jaws.

FIG. 5 illustrates a lateral view of the tool according to one preferred embodiment of the second aspect of the present invention, the tool comprising a clip with 3 jaws.

FIG. 6 illustrates a lateral view of the tool according to one preferred embodiment of the second aspect of the present invention, the tool comprising a catheter provided with an inflatable balloon.

FIG. 7 illustrates a lateral view of the tool according to one preferred embodiment of the second aspect of the present invention, the tool comprising a catheter comprising two conductor surfaces.

FIG. 8 illustrates a diagram of an example of equipment (current generator, cold water supply source, temperature measuring device, user interface, temperature controller . . . ) connected to the proximal extremity of the tool according to a second aspect of the present invention.

### DETAILED DESCRIPTION OF SEVERAL ASPECTS OF THE INVENTION

A first aspect of the invention concerns a needle preferably made of a composite material containing conductor elements—mixture of shape memory polymers and particles and/or electrical and/or thermic conductor (nano)fibers—having (at least) two stable shapes.

The first (stable) working shape is a rectilinear or straight shape.

The second (stable) working shape is a shape obligatory comprising a curved portion and optionally comprising a straight portion.

The curved portion is rigid, sharp, and its general shape is for example similar to an arc of circle.

The angle formed by the two radii joining the two extremities of the curved part is between about 120° and about 200°.

This shape can be equivalent to the needle of the “Ethilon® CPX 1 (45 mm)” or “Ethilon® FS-3 (16 mm ⅜ c)” type supplied by Ethicon® (Johnson&Johnson®).

Preferably, the radius of the curvature of the curved needle is between about 2.5 mm and about 22.5 mm.

Preferably, one extremity of the needle is sharp so as to easily penetrate the tissue.

Preferably, a surgical suture thread can be attached to the other extremity of the needle.

The flexural rigidity of a needle can be characterized by multiplying Young's modulus (E) of the material of the needle by the moment of inertia (I) of the straight section of the needle in relation to the neutral axis. Because of this, in order to obtain a flexural rigidity equivalent to the traditional needles for example of the “Ethilon® CPX 1 (45 mm)” or “Ethilon® FS-3 (16 mm ⅜ c)” type supplied by Ethicon® (Johnson&Johnson®), the product E I must be kept. It is therefore necessary to verify that:

EₐIₐ=E_{b}I_{b}

Eₐ: Young's modulus for a needle traditionally used for surgery.
Iₐ: Moment of inertia of the straight section of a needle, traditionally used for surgery, in relation to the neutral axis.
E_{b}: Young's modulus for the needle described in the present invention.
I_{b}: Moment of inertia of the straight section of the needle described in the present invention in relation to the neutral axis.

Preferably, the numerical value of the product E I is comprised in the interval between 0.1 10⁻³ Nm² and 10⁻² Nm².

Preferably, the diameter of the circle circumscribing the section of the needle will not exceed about 4 mm.

Still more preferably, the diameter of the circle circumscribing the section of the needle will not exceed about 3 mm.

The material making up the needle is a shape memory composite material that can comprise a multiblocks copolymer and any type of electrical and/or thermic conductor particles and/or fibers.

Preferably, the polymers are selected among biocompatible polymers.

The surface of one extremity of the needle is electrically and/or thermic conductive so as to quickly supply (or take away) calorific energy at the needle while allowing a temperature change by conduction of the entire needle.

Said surface is connected to the electrical and/or thermic conductive particles and/or fibers making up the needle.

Said surface is connected to the electrical and/or thermic conductive particles and/or fibers comprised in the needle.

Preferably, the surface of the other extremity of the needle can be conductive so as to quickly supply (or take away) calorific energy at the needle while allowing a temperature change by conduction of the entire needle.

Said surface is connected to the electrical and/or thermic conductive particles and/or fibers making up the needle.

Said surface is connected to the electrical and/or thermic conductive particles and/or fibers comprised in the needle.

The transition from the straight shape to the curved shape occurs when the temperature of the needle reaches a first switching temperature Ts₁ that is between about 38 and about 50 degrees Celsius.

Conversely, the transition from the curved needle to the straight needle occurs when the temperature of the needle reaches a second switching temperature Ts₂ that is between about 38 and about 50 degrees Celsius.

In this way, by controlling the temperature of one of the conductive surfaces, one can, owing to the conductive particles and/or fibers comprised in the composite material, control the shape of the needle and quickly modify it at any moment.

Advantageously, Ts₁=Ts₂=TS.

Preferably, the surface of the needle may be treated to obtain a low coefficient of friction and thereby facilitate penetration into the tissue.

The composite material used has a sufficient thermic conductivity for the change in temperature to occur in the entire needle within a maximum period of about 15 seconds, when the needle is heated or cooled.

Preferably, this period will be about 10 seconds at most.

Even more preferably, this period will be about 5 seconds at most.

A surgical suture thread traditionally used in surgery is preferably attached to one of the extremities of the needle.

Advantageously, this needle can be used as a diathermanous needle.

Advantageously, the needle according to the present invention can also be used by access routes other than flexible therapeutic endoscopy, such as through pediatric trocars, for example.

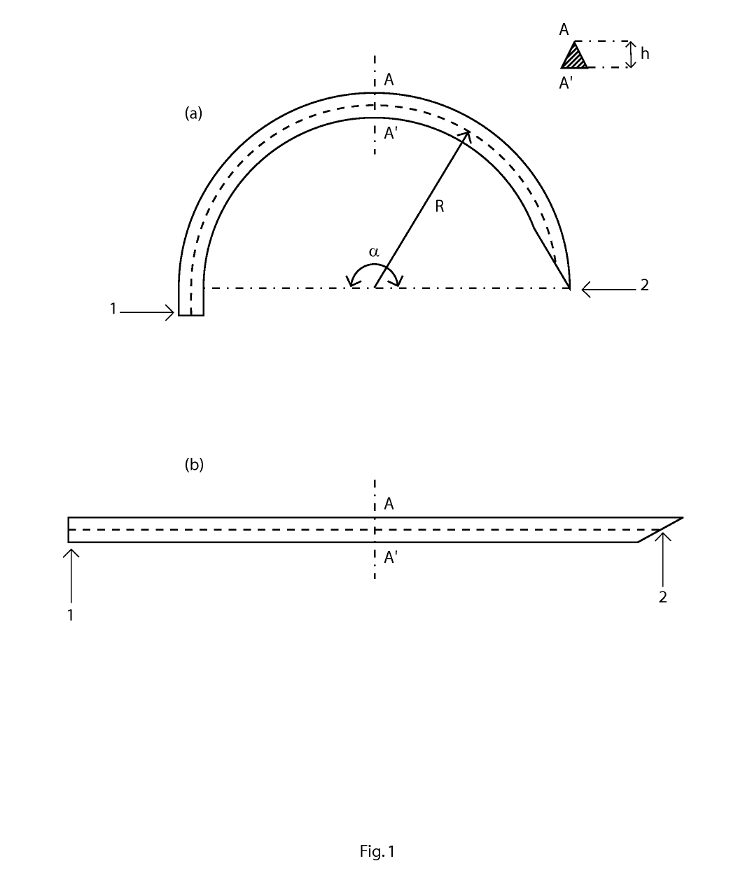

FIGS. 1a and 1b show the three stable shapes of the needle—curved shape (stable shape 2 illustrated in FIG. 1a) and straight shape (stable shape 1 and 3 illustrated in FIG. 1b)—according to a first preferred embodiment of the first aspect of the present invention.

In this preferred embodiment, the extremity 1 of the needle extends by a rectilinear segment.

The switching temperature between these shapes is about 40° C.

The radius R of the curved shape of the needle is about 12.5 mm.

The angle α formed by the radii joining the two extremities of the curved part of this needle is about 180°.

The section of the needle is an equilateral triangle with a height h equal to about 1.6 mm.

The needle comprises conductive fibers submerged in a shape memory polymer material.

The conductive fibers are connected to the two conductive surfaces situated at the two extremities of the needle.

At its extremity 2, the needle thins so as to form a bevel.

A surgical wire is attached at its extremity 1.

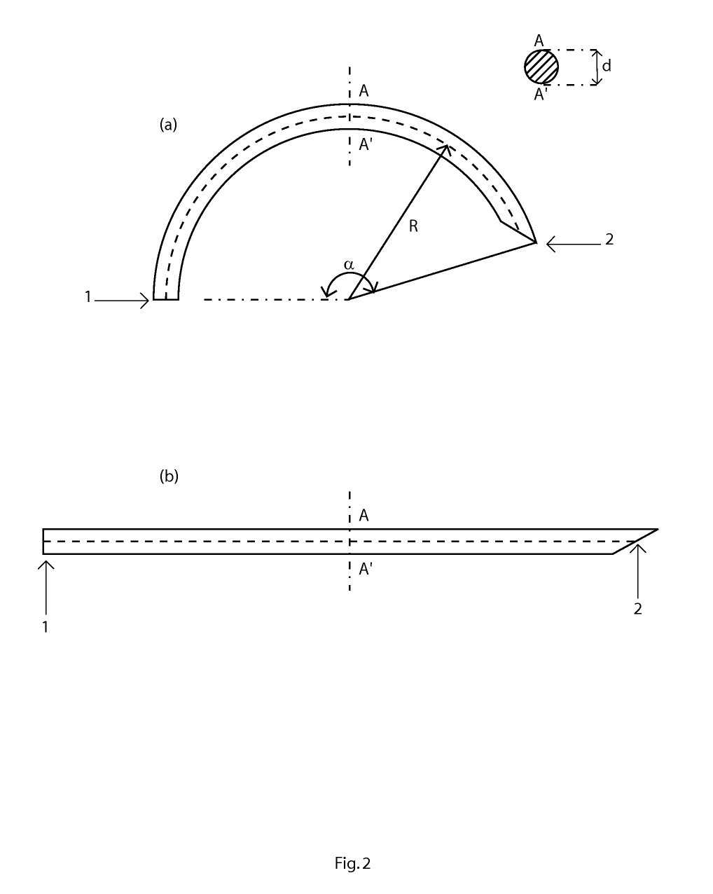

FIGS. 2a and 2b show the three stable shapes of the needle—curved shape (stable shape 2 illustrated in FIG. 2a) and straight shape (stable shape 1 and 3 illustrated in FIG. 2b)—according to a second preferred embodiment of the first aspect of the present invention.

In this preferred embodiment, the extremity 1 of the needle does not extend by a rectilinear segment.

The extremity 2 of the needle is beveled sharp.

The switching temperature between these shapes is about 45° C.

The radius R of the curved shape of the needle is about 20 mm.

The angle α formed by the radii joining the two extremities of the curved part of said needle is about 160°.

The section of the needle is a circle whereof the diameter d is about 2.8 mm.

The needle is made of a memory shape polymer material containing a co-continuous distribution of particles and/or conductive fibers.

At its extremity 2, the needle thins so as to form a point.

A surgical wire is attached at its extremity 1.

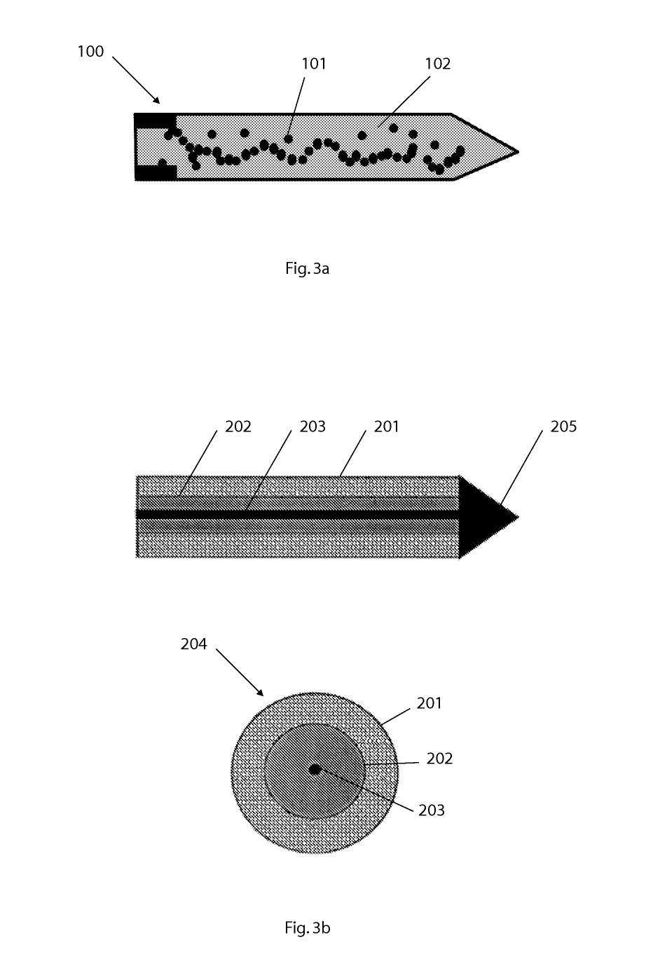

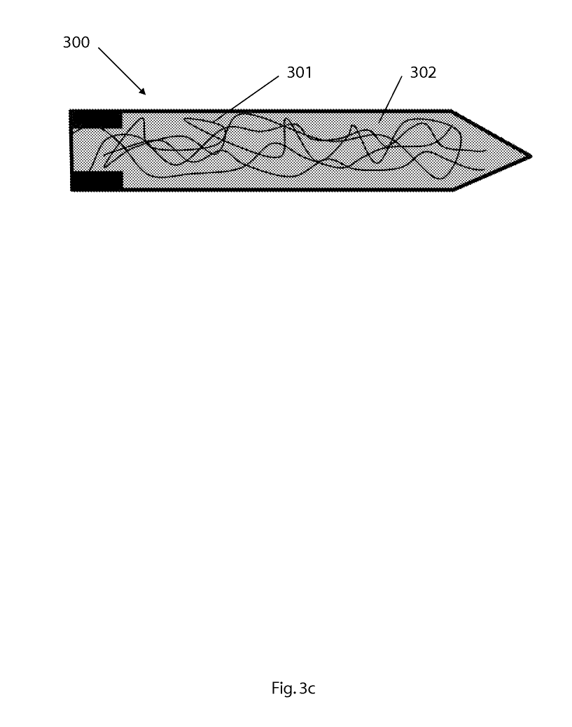

As shown in FIG. 3a, the needle 100 can also assume the form of a shape memory polymer matrix 102 comprising isotropic conductive charges 101 (e.g. carbon blacks).

As illustrated in FIG. 3b, the needle 301 can also assume the form of a shape memory polymer matrix 302 comprising conductive fibers (e.g. nanotubes).

As illustrated in FIG. 3, the needle can also assume the form of a profile of coextruded polymers comprising at least two layers of polymers 201, 202, the layers having distinct softening temperatures.

Preferably, a first polymer layer has a low softening temperature and a second polymer layer has a high softening temperature.

Preferably, the softening temperature of the second polymer is at least 10° C. higher than that of the first polymer.

Preferably, the softening temperature of the second polymer is at least 20° C. higher than the softening temperature of the first polymer.

Advantageously, the coextruded layers assume a concentric form.

Preferably, the layer having the lowest softening temperature is an inner or intermediate layer of the profile.

Advantageously, the profile in addition comprises a conductive core 203, formed by a metallic wire or a layer of a conductive polymer composition material.

Preferably, another layer of the profile 201, insulated from the conductive core, is conductive, forming a coaxial conductor 204.

Preferably, at the head of the needle, the core layer 203 and the second conductive layer can be connected so as to form a circuit.

Preferably, at the tail of the needle, said layers are connected to the current supply [supply?] so as to be able to generate heating by Joule effect.

Preferably, the connection between the conductive layers 201, 203 at the head of the needle comprises a conductive metallic insert 205.

Advantageously, the insert assumes a sharp form.

The needle can for example be produced as follows:
extruding, using a die having the desired needle profile, a polyphase copolymer, having separate phases that can be co-continuous, at a temperature higher than the switching temperature of the phase having the highest melting temperature,
forming the snap ring directly in its curved shape at the desired radius of curvature in the curved shape at a temperature higher than the switching temperature of the phase having the highest switching temperature;
solidifying the snap ring in its curved shape by bringing it to a temperature situated between the switching temperature of the phase having the highest melting temperature and the switching temperature of the phase having the lowest melting temperature;
forming the snap ring in its straight shape by applying an external mechanical stress and cooling it at a temperature below the switching temperature of the phase having the lowest switching temperature. The external mechanical stress is then removed and the straight snap ring is obtained;
cutting the straight snap ring to the desired length and sharpening it to obtain a needle.

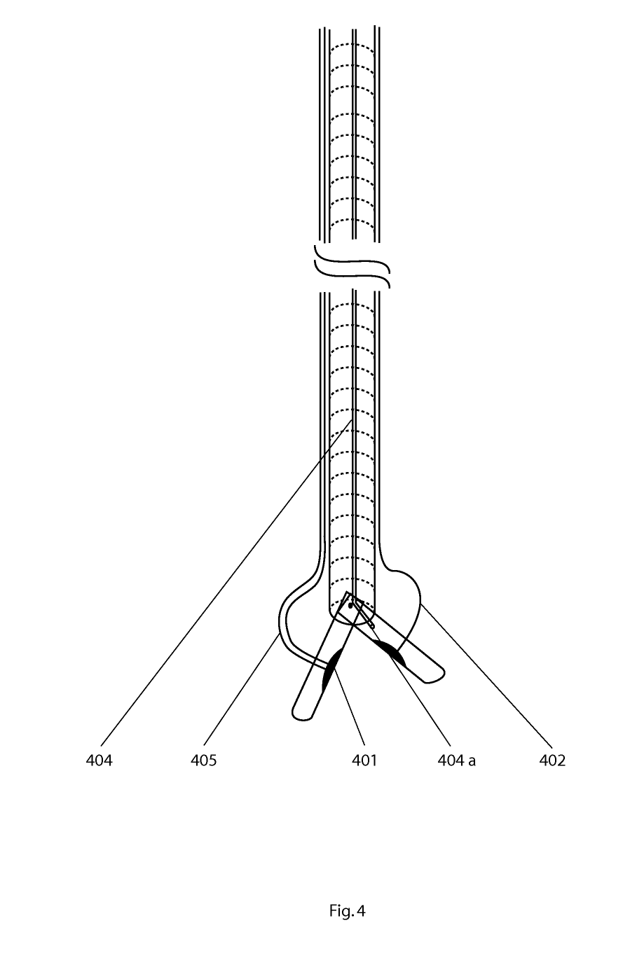

FIG. 4 describes an endoscopic clip with two jaws making it possible to grasp a foreign body situated in the human body according to one embodiment of the second aspect of the present invention. Each of the jaws contains, on its inner portion, a conductive surface (401) insulated from the rest of the clip. Welded to each conductive surface is a conductive wire (402) that is connected to a current generator (406) situated outside the patient's body. The current loop is closed by the foreign body.

The outlet (404a) of the fluid intake tube (404) is oriented on one of the conductive parts (401) of the clip.

The other extremity of the tube is connected to a reservoir (409) through a pump (407) controlled by a controller (408).

A thermocouple (405) is placed on one of the conductive parts and is connected to a temperature measuring device (1) linked to a temperature control system (411) connected to a user interface (412).

The user interface allows the user to enter the wanted temperature stting point and to view the temperature measured by the thermocouple (405).

The controller (411) makes it possible to regulate the temperature of the conductive parts of the clip by acting on the controller (408) controlling the pump (407) and/or on the current generator (406) as described in FIG. 8.

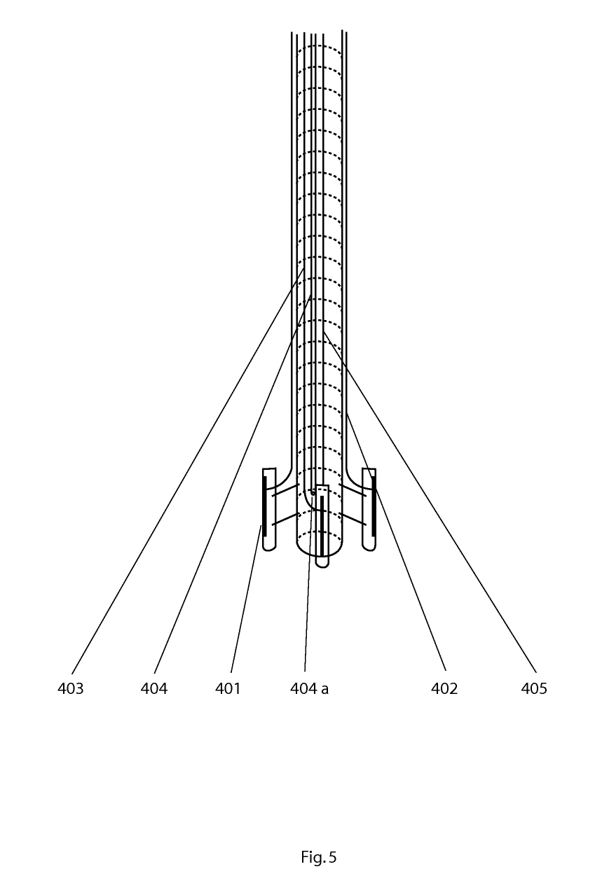

FIG. 5 describes a clip with three jaws making it possible for example to hold a cylinder by its inner surface according to one embodiment of the second aspect of the present invention.

Each of the jaws contains, on its outer part, a conductive surface (401) insulated from the rest of the clip. Welded on two of these conductive surfaces is a conductive wire (402) that is connected to a current generator (406) situated outside the patient's body.

The current loop is closed by the foreign body. The outlet (404a) of the fluid intake tube (404) is oriented on one of the conductive parts (401) of the clip.

The other extremity of the tube is connected to a reservoir (409) through a pump (407) controlled by a controller (408).

A thermocouple (405) is placed on one of the conductive parts and is connected to a temperature measuring device (410) linked to a temperature control system (411) connected to a user interface (412).

The user interface allows the user to enter the desired temperature value and view the temperature measured by the thermocouple (405).

The controller (411) makes it possible to regulate the temperature of the conductive portions of the clip by acting on the controller (408) controlling the pump (407) and/or on the current generator (406) as described in FIG. 8.

Connected to the third conductive portion is a conductive wire (403) making it possible to provide the necessary energy to the foreign body to be able to perform electrocoagulation diathermy.

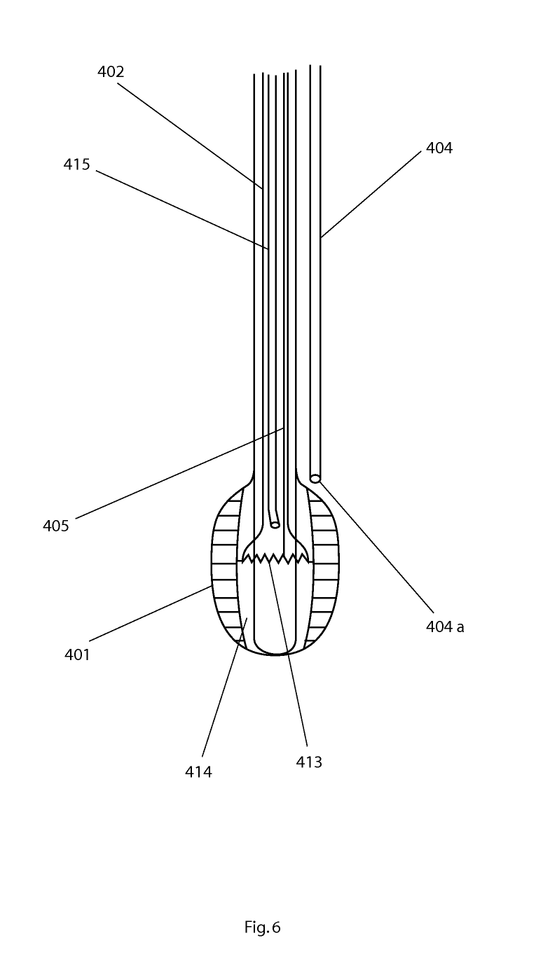

FIG. 6 describes a catheter provided with an inflatable balloon (114) according to one embodiment of the second aspect of the present invention.

The balloon (414) can be inflated by a fluid coming from the tube (415). The balloon (414) makes it possible for example to hold a cylinder by its inner surface. The balloon (414) is provided with two conductive surfaces (401) connected by a heating resistance (413), on its outer portion, insulated from the rest of the catheter. Welded on each of its conductive surfaces (401) is a conductive wire (402) connected to a current generator (406) situated outside the patient's body.

The outlet (404a) of the fluid intake tube (404) is oriented on one of the conductive portions (401) of the balloon. The other extremity of the tube is connected to a reservoir (409) via a pump (407) controlled by a controller (408).

A thermocouple (405) is placed on the heating resistance (413) and is connected to a temperature measuring device (410) linked to a temperature control system (411) connected to a user interface (412).

The user interface allows the user to enter the desired temperature value and view the temperature measured by the thermocouple (405).

The controller (411) makes it possible to regulate the temperature of the conductive portions (401) of the balloon (414) by acting on the controller (408) controlling the pump (407) and/or on the current generator (406) as described in FIG. 8.

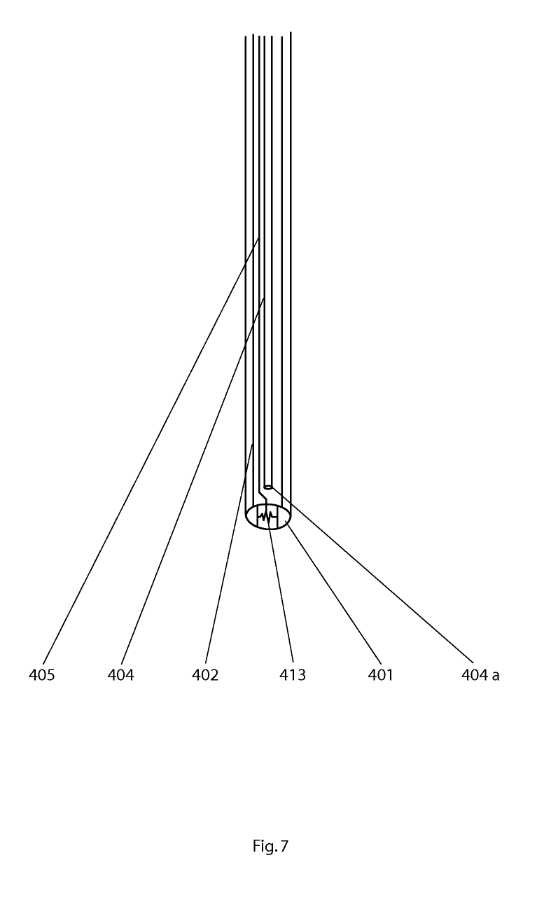

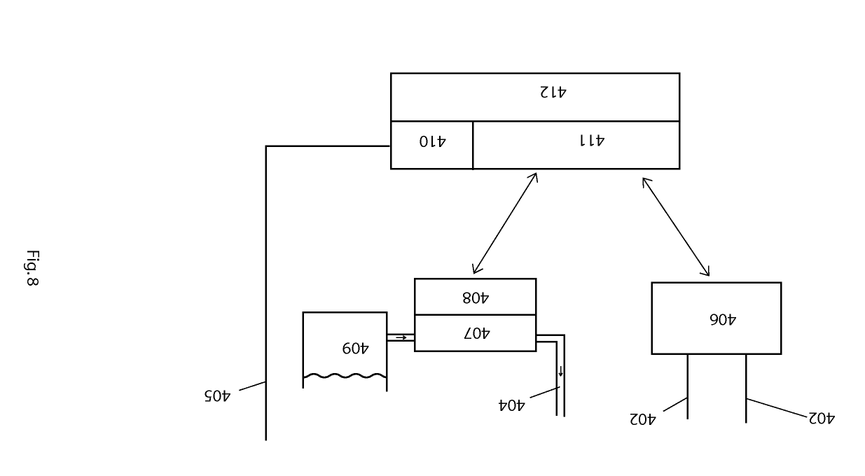

FIG. 7 describes a catheter whereof the distal extremity surface comprises two conductive surfaces (401) connected by a heating resistance (413) insulated from the rest of the catheter according to one embodiment of the second aspect of the present invention.

Welded to the extremities of this heating resistance are two conductive wires (402) that are connected to a current generator (406) situated outside the patient's body.

The outlet (404a) of the fluid intake tube (404) is oriented on the heating resistance (413).

The other extremity of the tube is connected to a reservoir (409) through a pump (407) controlled by a controller (408).

A thermocouple (405) is placed on the heating resistance (413) and is connected to a temperature measuring device (410) linked to a temperature control system (411) connected to a user interface (412). The user interface allows the user to enter the wanted temperature setting point and to visualize the temperature measured by the thermocouple (405).

The controller (411) allows a temperature regulation on the heating resistance (413) by acting on the pump (407) and/or on the current generator (406) as described in FIG. 8.

### Examples

### 1. Transmural Gastric Plication

The needle shown in FIGS. 1a and 1b was used to perform a transmural gastric plication on a pig stomach ex vivo.

Said needle being inserted into the endoscope, in straight form, the experimenter pushes it through the wall situated several centimeters below the line Z.

Once it is two-thirds inserted through the gastric wall, it is heated so as to bend to 180°.

The needle is then removed with the help of the endoscope and its extremity 2 was grasped by a clip introduced into the second operator channel.

After this first passage is done, the needle is cooled, returned, reinserted through the wall of the stomach, and heated again to find its curve at 180°.

This maneuver is done three times in a row in order to obtain a triple plication of the stomach.

The needle was then cooled and recovered straight through the operator channel of the endoscope.

A suture has been performed on the 2 strands of the thread attached to the needle.

### 2. Overhand Gastric Suture

The needle shown in FIGS. 1a and 1b was used to perform a overhand gastric plication on a pig stomach ex vivo.

First, said needle is inserted in its straight form into the rear face of the antrum.

It is then heated to curve at 180°, recovered with the second operator channel of the endoscope, after having passed back through, from the outside to the inside, the gastric wall.

The needle and its thread are then brought back over a length of about 25 cm in the gastric cavity and the 2 strands emerging in the gastric wall are solidarized through the second operator channel.

The needle is then cooled, reinserted in the anterior wall of the stomach, reheated, recovered in the gastric cavity, cooled, reinserted into the posterior wall of the stomach, reheated and recovered in the gastric cavity, and so on to obtain a overhand suture with five or six sutures, making it possible to affix the posterior face of the stomach to its anterior face.

At the extremity of the overhand suture, the needle is recovered in the gastric cavity, curved under the effect of the heat and inserted between the loops of the last overhand suture, in order to make a surgical double knot.

### 3. NOTES Model

In a NOTES (Natural Orifice Transluminal Endoscopic Surgery) model, on a living animal, an iatrogenic perforation of the gastric cavity is made with the help of a diathermanous needle and an expansion of the balloon of about 18 mm.

The endoscope is inserted into the peritoneum and, when it is removed, the needle, described in FIGS. 1a and 1b, is inserted in its straight shape into the endoscope, the gastric wall having been punctured.

At that moment, the needle is heated to curve at 180°.

Its extremity then punctures the contralateral outer wall of the stomach, in relation to the orifice.

The needle is recovered in the gastric cavity and then cooled.

A new puncture is done, staggered by 90° in relation to the preceding one, with the needle in its straight shape.

The needle is then reheated, recovered in the gastric cavity and, on both of the strands resulting from that cross-shaped point, a suture is applied.

### 4. Rigid Endoscopy Suture

The needle shown in FIGS. 1a and 1b is inserted in its straight shape, in the peritoneum of a living animal, through a pediatric trocar. Once in the peritoneum, it is heated to curve at 180°.

We then have a curved needle in the peritoneum that can be used as a traditional surgical needle to perform a suture. Once the suture is made, the needle is cooled to return to its straight shape. It is thus removed from the peritoneal cavity through the pediatric trocar.

If we have a straight needle that can be inserted into a pediatric trocar and that has the particularity of being made from a material changing its configuration at higher temperature (for example above the maximum temperature of the human body (>40° C.)), the straight needle can be grasped by the present invention, inserted straight into the peritoneal cavity through the pediatric trocar, and heated via the present invention, which causes its elasticity. We then have a curved needle in the peritoneal cavity that can be used as a traditional surgical needle to perform a suture. Once the suture is done, the needle is cooled via the tool according to the second aspect of the present invention to return to its straight shape and can thus be removed from the peritoneal cavity through the pediatric trocar.

### FIGURES

| French | English |
|---|---|
| Feuille de remplacement (règle 26) | Replacement sheet (rule 26) |

1.-75. (canceled)

76. A shape memory surgical needle comprising a needle body:
having two extremities, one proximal extremity and one sharp distal extremity,
said body having at least two different stable shapes, a first shape stable at a first temperature range and a second shape stable at a second temperature range, the first temperature range being strictly lower than the second temperature range,
wherein:
the material making up the body of the needle comprises at least one shape memory insulating material and at least one conductor material or one thermic and/or electrical conductor charge,
the entire body of the needle being capable of being heated or cooled.

77. The surgical needle according to claim 76, wherein the entire body of the needle is heated by applying a heat and/or current supply to obtain a switch from the first to the second stable form.

78. The surgical needle according to claim 76, wherein the insulating material comprises a polymer matrix, and in particular a multiblocks copolymer comprising at least two types of blocks defining two polymers.

79. The surgical needle according to claim 78, wherein the first type of block has a low softening temperature and the second type of block has a high softening temperature.

80. The surgical needle according to claim 79, wherein the softening temperature of the first type of block is lower than the softening temperature of the second type of block.

81. The surgical needle according to claim 79, wherein the softening temperature is either a vitreous transition temperature or a melting temperature.

82. The surgical needle according to claim 78, wherein each of the types of block has at least two terminal alcohol functions.

83. The surgical needle according to claim 78, wherein the multiblocks are obtained as a reaction product of several blocks each comprising at least two terminal alcohol functions with diisocyanates.

84. The surgical needle according to claim 78, wherein the polymer matrix results from the reaction of a first polymer and a second polymer with a diisocyanate in which both the first and the second polymers comprise terminal alcohol functions.

85. The surgical needle according to claim 78, wherein the softening temperature of the first polymer is between 40 and 45° C.

86. The surgical needle according to claim 78, wherein the softening temperature of the first polymer is the switching temperature between two stable shapes of the needle.

87. The surgical needle according to claim 78, wherein the softening temperature of the second polymer is at least 20° C. higher than that of the first polymer.

88. The surgical needle according to claim 78, wherein the first polymer and the second polymer form separate phases.

89. The surgical needle according to claim 78, wherein the two polymers are selected from the group consisting of aliphatic polyesters, aliphatic polyester-alt-ethers, aliphatic polycarbonates, poly(ethylene glycol) and all combinations thereof.

90. The surgical needle according to claim 76, wherein the thermic and/or electrical conductor charge assumes the form of powder, fiber or sheets with dimensions between about 10 μm and 1 nm.

91. The surgical needle according to claim 76, wherein the conductive charge is selected from the group consisting of carbon blacks, carbon fibers, carbon nanofibers, carbon nanotubes, grapheme lamellas (exfoliated graphite), or a mixture thereof.

92. The surgical needle according to claim 76, wherein the entire body of the needle is cooled by applying a cold source to it.

93. The surgical needle according to claim 92, wherein the temperature variation caused by the cold source in the needle allows the transition from the second stable shape at a second temperature range to a third stable shape at a third temperature range.

94. The surgical needle according to claim 93, wherein the third stable shape corresponds to the first stable shape.

95. The surgical needle according to claim 76, wherein the transition between said first and second shapes occurs at a switching temperature between a minimum temperature corresponding to a temperature just above body temperature, at a switching temperature between 40° C. and 45° C.

96. The surgical needle according to claim 76, wherein the distal extremity of the needle has a metallic or ceramic insert making it possible to ensure the integrity of its sharpness.
